(19) United States
(12) Patent Application Publication
(10) Pub. No.: US 2012/0100194 A1
(43) Pub. Date: Apr. 26, 2012

(54) CHEWING GUM COMPOSITION, CHEWING GUM PRODUCT, AND METHOD FOR MANUFACTURING SAME

(75) Inventors: Masafumi Yamai, Tokyo (JP); Yoshiaki Adachi, Tokyo (JP); Samantha Holme, Pompton Plains, NJ (US); Kenichiro Yanagi, Tokyo (JP)

(73) Assignee: KRAFT FOODS GLOBAL BRANDS LLC, Northfield, IL (US)

(21) Appl. No.: 13/266,213

(22) PCT Filed: Apr. 30, 2010
(86) PCT No.: PCT/US10/33176

§ 371 Date: Dec. 20, 2011

(30) Foreign Application Priority Data
Apr. 30, 2009 (JP) 2009-111071

(51) Int. Cl. A61K 8/02; A61Q 11/00; A61P 1/02; A61K 9/68

(52) U.S. Cl. 424/401; 424/48

(57) ABSTRACT

The invention relates to a chewing gum composition, a chewing gum product, and a method for manufacturing the same where the chewing gum composition includes: a gum base; and a gingivitis-inhibiting amount of hyaluronic acid or a physiologically acceptable salt thereof.

### TECHNICAL FIELD

The present invention relates to a chewing gum composition, a chewing gum product, and a method for manufacturing the same.

### BACKGROUND ART

Conventionally, a treatment agent using hyaluronic acid along with an anti-inflammatory agent is known as a means for suppressing inflammation of mucous membranes. For example, Japanese Translation of PCT International Publication No. Hei 11-514967 discloses that an oral tumor is effectively suppressed by applying a gel containing a nonsteroidal anti-inflammatory agent and hyaluronic acid onto an affected area. Furthermore, the dosage form of the conventional treatment agent is not limited to a gel. A mouth rinse, a spray, a dentifrice and the like are also known as conventional treatment agents.

### DISCLOSURE OF THE INVENTION

### Problems to be Solved by the Invention

However, the abovementioned dosage forms require applying or atomizing the treatment agent directly onto an affected area, and are bothersome. This makes continued treatment difficult and, consequently, there have been many cases where satisfactory treatment effects cannot be obtained.

To avoid a bothersome procedure such as applying and atomizing, a beverage or food containing hyaluronic acid and the like may be used. However, most beverages and foods are masticated in a short time, and thus an active ingredient such as hyaluronic acid does not stay on the affected area in the oral cavity and quickly flows out to the stomach. Therefore, there are many cases where a satisfactory treatment effect cannot be obtained.

The present invention is proposed in view of the above-mentioned circumstances and aims at providing a means for satisfactorily suppressing gingivitis that can provide hyaluronic acid to an affected area continuously and persistently, with a minimum of psychological stress.

### Means for Solving the Problems

The present inventors found that, among other beverages and foods, chewing gum in particular has superior sustained-release properties of hyaluronic acid and can provide an effective dosage of hyaluronic acid to every part of the oral cavity over an extended time period, and thus arrived at the completion of the present invention. More specifically, the present invention provides the following.

In a first aspect of the present invention, a chewing gum composition includes: a gum base; and a gingivitis-inhibiting amount of hyaluronic acid or a physiologically acceptable salt thereof.

According to a second aspect of the present invention, in the chewing gum composition as described in the first aspect, the hyaluronic acid has an average molecular weight of at least 400 and no greater than 10000000.

According to a third aspect of the present invention, in the chewing gum composition as described in the first or the second aspect, the gingivitis-inhibiting amount is at least 0.0075% by mass and no greater than 95% by mass with respect to a mass of the entire composition.

According to a fourth aspect of the present invention, in the chewing gum composition as described in any one of the first to the third aspects, the gum base contains fat and oil in an amount of at least 20% by mass with respect to a mass of the entire gum base.

According to a fifth aspect of the present invention, in the chewing gum composition as described in any one of the first to the fourth aspects, the gum base is substantially free from a wax.

According to a sixth aspect of the present invention, the chewing gum composition as described in any one of the first to the fifth aspects further includes: at least one sweetener; and casein phosphopeptide-calcium phosphate complex in an amount of at least 0.01% by mass and no greater than 95% by mass with respect to the mass of the composition.

According to a seventh aspect of the present invention, in the chewing gum composition as described in the sixth aspect, at least a part of the casein phosphopeptide-calcium phosphate complex is encapsulated.

According to an eighth aspect of the present invention, in the chewing gum composition as described in the sixth or the seventh aspect, the sweetener includes at least one sugarless sweetener.

According to a ninth aspect of the present invention, the chewing gum composition as described in any one of the first to the eighth aspects further includes at least one flavor.

According to a tenth aspect of the present invention, a chewing gum product includes the chewing gum composition as described in any one of the first to the ninth aspects.

According to an eleventh aspect of the present invention, the chewing gum product as described in the tenth aspect includes at least 0.2 mg and no greater than 560 mg of hyaluronic acid or a physiologically acceptable salt thereof.

According to a twelfth aspect of the present invention, the chewing gum product as described in the tenth or the eleventh aspect is plate-like, slab, powdered, liquid, paste, pellet, rod-like, center-filled, deposited, or pressed.

According to a thirteenth aspect of the present invention, the chewing gum product as described in any one of the tenth to the twelfth aspect includes: a central filled region; and a coating region that at least partially coats the central filled region.

According to a fourteenth aspect of the present invention, in the chewing gum product as described in the thirteenth aspect, the hyaluronic acid or the physiologically acceptable salt thereof is disposed in the central filled region, the coating region, or in both thereof.

According to a fifteenth aspect of the present invention, the chewing gum product as described in any one of the tenth to the fourteenth aspect includes at least 0.1 mg of casein phosphopeptide-calcium phosphate complex.

According to a sixteenth aspect of the present invention, the chewing gum product as described in any one of the tenth to the fifteenth aspects includes an indicia indicating effectiveness for therapy, prophylaxis, or retardation of progress of gingivitis.

In a seventeenth aspect of the present invention, a preparation method for a chewing gum composition for inhibiting gingivitis includes a step of blending a gum base with a gingivitis-inhibiting amount of hyaluronic acid or a physiologically acceptable salt thereof.

According to an eighteenth aspect of the present invention, in the preparation method as described in the seventeenth aspect, the hyaluronic acid or the physiologically acceptable salt thereof is added in a last half of a process of blending other components.

According to a nineteenth aspect of the present invention, in the preparation method as described in the seventeenth or the eighteenth aspect, the hyaluronic acid used has an average molecular weight of at least 400 and no greater than 10000000.

According to a twentieth aspect of the present invention, in the preparation method as described in any one of the seventeenth to the nineteenth aspects, the gingivitis-inhibiting amount is at least 0.0075% by mass and no greater than 95% by mass with respect to amass of the entire composition.

According to a twenty-first aspect of the present invention, in the preparation method as described in any one of the seventeenth to the twentieth aspects, fat and oil is blended in a ratio of at least 20% by mass with respect to a mass of the entire gum base.

According to a twenty-second aspect of the present invention, in the preparation method as described in any one of the seventeenth to twenty-first aspects, a wax is substantially not blended into the gum base.

According to a twenty-third aspect of the present invention, the preparation method as described in any one of the seventeenth to the twenty-second aspects further blends: at least one sweetener; and casein phosphopeptide-calcium phosphate complex in a ratio of at least 0.01% by mass and no greater than 95% by mass with respect to the mass of the composition.

According to a twenty-fourth aspect of the present invention, in the preparation method as described in the twenty-third aspect, at least a portion of the casein phosphopeptide-calcium phosphate complex is encapsulated.

According to a twenty-fifth aspect of the present invention, in the preparation method as described in the twenty-third or the twenty-fourth aspect, the sweetener includes at least one sugarless sweetener.

According to a twenty-sixth aspect of the present invention, the preparation method as described in any one of the seventeenth to the twenty-fifth aspects further blends at least one flavor.

According to a twenty-seventh aspect of the present invention, a preparation method for a chewing gum product uses the chewing gum composition for inhibiting gingivitis as described in any one of the first to the ninth aspects.

According to a twenty-eighth aspect of the present invention, in the preparation method as described in the twenty-seventh aspect, a content of hyaluronic acid or a physiologically acceptable salt thereof is at least 0.2 mg and no greater than 560 mg.

According to a twenty-ninth aspect of the present invention, the preparation method as described in the twenty-seventh or twenty-eighth aspect produces a plate-like shape, a slab shape, a powdered form, a liquid form, a paste form, a pellet form, a rod-like shape, a center-filled form, a deposited form, or a pressed form.

According to a thirtieth aspect of the present invention, the preparation method as described in any one of the twenty-seventh to twenty-ninth aspects provides: a central filled region; and a coating region that at least partially coats the central filled region.

According to a thirty-first aspect of the present invention, in the preparation method as described in the thirtieth aspect, the hyaluronic acid or the physiologically acceptable salt thereof is disposed in the central filled region, the coating region, or in both thereof.

According to a thirty-second aspect of the present invention, in the preparation method as described in any one of the twenty-seventh to thirty-first aspects, a content of casein phosphopeptide-calcium phosphate complex is at least 0.1 mg.

According to a twenty-third aspect of the present invention, the preparation method as described in any one of the twenty-seventh to thirty-second aspects includes providing a notice indicating effectiveness for therapy, prophylaxis, or retardation of progress of gingivitis.

In a thirty-fourth aspect of the present invention, a kit for therapy or prophylaxis for gingivitis in a mammal includes: a chewing gum product containing a gum base, and hyaluronic acid or a physiologically acceptable salt thereof; an instructional insert for the chewing gum product; and a package for encasing the chewing gum product and the instructional insert.

In a thirty-fifth aspect of the present invention, a kit for therapy or prophylaxis for gingivitis in a mammal including: a chewing gum product containing a gum base, and hyaluronic acid or a physiologically acceptable salt thereof; an instructional insert for the chewing gum product; and a package for encasing the chewing gum product and the instructional insert.

### EFFECTS OF THE INVENTION

According to the present invention, since a gum base is used along with a gingivitis-inhibiting amount of hyaluronic acid or a physiologically acceptable salt thereof, hyaluronic acid is released in a controlled manner when masticated in the oral cavity. This provides an effective dosage of hyaluronic acid in the oral cavity over an extended time period. In addition, mastication stimulates the secretion of saliva that can bring hyaluronic acid to every part in the oral cavity, and biological substances in saliva are expected to disinfect or inactivate germs. Furthermore, the abovementioned effect is obtained only by masticating a chewing gum and hyaluronic acid can be provided to an affected area continuously and persistently, with a minimum of psychological stress. Therefore, an effect of suppressing gingivitis can be synergistically obtained.

### PREFERRED MODE FOR CARRYING OUT THE INVENTION

An embodiment of the present invention is described hereinafter; however, the present invention is not limited thereto.

Chewing Gum Composition and Chewing Gum Product

A chewing gum composition according to the present invention contains a gingivitis-inhibiting amount of hyaluronic acid or a physiologically acceptable salt thereof and a gum base. Each of the ingredients is described in detail hereinafter.

Hyaluronic Acid or Physiologically Acceptable Salt Thereof

A chewing gum composition according to the present invention contains a gingivitis-inhibiting amount of hyaluronic acid or a physiologically acceptable salt thereof. As used herein, “gingivitis-inhibiting amount” is a sufficient amount with which gingivitis is at least partially prevented or cured, or progression thereof is retarded by a single instance or multiple instances of mastication of the chewing gum. Thus far, although a beverage and food containing hyaluronic acid are conventionally available, an intended purpose thereof is skin-care, and thus the content of hyaluronic acid is set for a case where hyaluronic acid is carried from the oral cavity to the stomach, and then absorbed in the body. On the other hand, the gingivitis-inhibiting amount is set so that a predetermined amount of hyaluronic acid stays within the oral cavity over an extended time period, thus being completely different from a standard content of hyaluronic acid for conventional beverages and food.

The gingivitis-inhibiting amount is not particularly limited and can be arbitrarily set in accordance with material of the gum base, molecular weight of hyaluronic acid, a degree of gingivitis to be addressed, and the like.

According to an embodiment, the gingivitis-inhibiting amount may be no greater than 600 mg, no greater than 550 mg, no greater than 500 mg, no greater than 450 mg, no greater than 400 mg, no greater than 350 mg, no greater than 300 mg, no greater than 250 mg, no greater than 200 mg, no greater than 150 mg, no greater than 100 mg, no greater than 50 mg, no greater than 40 mg, no greater than 20 mg, no greater than 15 mg, no greater than 10 mg, no greater than 5 mg, no greater than 1 mg, no greater than 0.5 mg, or no greater than 0.1 mg per serving (per one intake). According to another embodiment, the gingivitis-inhibiting amount can be 0.1 to 600 mg, 0.2 to 560 mg, 0.2 to 200 mg, 0.5 to 40 mg, 1 to 15 mg, and 2 to 10 mg. Here, 1 serving may consist of 1 to 8 pieces of the product, namely 7, 6, 5, 4, 3, or 2 pieces, although not particularly limited thereto. In addition, the mass of each product may be in a range of 1 to 40 g, namely 30 g, 20 g, 10 g, 8 g, 6 g, 4 g, 2 g, 1 g, or 0.5 g, although not particularly limited thereto.

According to an embodiment, the gingivitis-inhibiting amount may be no greater than 600 mg, no greater than 400 mg, no greater than 200 mg, no greater than 150 mg, no greater than 100 mg, no greater than 50 mg, no greater than 40 mg, no greater than 30 mg, no greater than 25 mg, no greater than 10 mg, no greater than 8 mg, no greater than 5 mg, or no greater than 1 mg per day. According to an embodiment, the gingivitis-inhibiting amount may be in a range of 5 to 600 mg, 25 to 300 mg, 5 to 50 mg, or 8 to 40 mg per day. Here, the amount per day may include a desired arbitral number of servings or products.

According to an embodiment, the gingivitis-inhibiting amount can be no greater than 95%, no greater than 90%, no greater than 85%, no greater than 80%, no greater than 75%, no greater than 70%, no greater than 65%, no greater than 60%, no greater than 55%, no greater than 50%, no greater than 45%, no greater than 40%, no greater than 35%, no greater than 30%, no greater than 25%, no greater than 20%, no greater than 15%, no greater than 10%, no greater than 5%, no greater than 2%, no greater than 1%, no greater than 0.5%, no greater than 0.2%, no greater than 0.1%, no greater than 0.01%, or no greater than 0.001% with respect to the mass of the product. According to another embodiment, the gingivitis-inhibiting amount may be in a range of 0.001 to 95%, 0.0075 to 80%, 0.01 to 60%, 0.01 to 10%, 0.01 to 5%, 0.02 to 5%, or 0.01 to 2% with respect to the mass of the product.

In the present specification, “hyaluronic acid” designates polysaccharides having at least one repeating unit composed of a disaccharide of glucuronic acid and N-acetylglucosamine. In addition, “physiologically acceptable salt” is not particularly limited as long as it is harmless to administer to the human body, and includes alkali metal salt (for example, sodium salt and potassium salt), alkaline earth metal salt (for example, calcium salt and magnesium salt), zinc salt, ammonium salt and the like.

An average molecular weight of hyaluronic acid is preferably at least 500 and more preferably at least 200000, since, in a case where the average molecular weight is insufficient, the property for a controlled release becomes poor and it is difficult to provide a sufficient amount of hyaluronic acid to an affected area continuously. On the other hand, the average molecular weight of hyaluronic acid is preferably no greater than 10000000, more preferably no greater than 800000, and most preferably no greater than 2000000 since, in a case where the average molecular weight is excessive, a sufficient amount of hyaluronic acid is not released and it is difficult to provide a sufficient amount of hyaluronic acid to an affected area.

The average molecular weight herein is a molecular weight calculated from an intrinsic viscosity of hyaluronic acid or a salt thereof. To obtain an intrinsic viscosity of hyaluronic acid or salt thereof, first, a plurality of solution of hyaluronic acid or a salt thereof is prepared, and a specific viscosity and reduced viscosity are obtained from efflux time (seconds) of the solution of hyaluronic acid or a salt thereof and of a solvent determined by an Ubbelohde viscometer (manufactured by Shibata Scientific Technology Ltd.), in accordance with the following equations (1) and (2).

Specific Viscosity=(Efflux Time (Seconds) of Solution of Hyaluronic Acid or Salt Thereof/Efflux Time (Seconds) of Solvent)−1 Equation 1

Reduced Viscosity=Specific Viscosity/Concentration of Hyaluronic Acid or Salt Thereof in Terms of Dry Matter (g/100 ml) Equation 2

The Ubbelohde viscometer used herein has a factor that makes the efflux time 200 to 1000 seconds. The concentration of the solution of hyaluronic acid or salt thereof is selected within an appropriate range for a measuring instrument. A measurement is carried out in a thermobath of 30° C. so that no temperature change occurs.

Next, a calibration curve is obtained for each solution of hyaluronic acid or salt thereof, by plotting the reduced viscosity obtained on a vertical axis and the concentration of hyaluronic acid or salt thereof in terms of dry matter on a horizontal axis. Then, the intrinsic viscosity of hyaluronic acid or salt thereof is obtained by extrapolating the concentration of hyaluronic acid or salt thereof to 0. Furthermore, an average molecular weight M can be obtained from the intrinsic viscosity of hyaluronic acid or salt thereof in accordance with the following equation 3.

Intrinsic Viscosity (dl/g)=K′Mα Equation 3

(wherein K′=0.036 and α=0.78)

It should be noted that the average molecular weight of hyaluronic acid can be a molecular weight obtained by well-known measurement methods using gel filtration, ultrafiltration and the like. Hyaluronic acid or a salt thereof is known to be precipitated being combined with CPC (cetylpyridinium chloride). Therefore, the presence of hyaluronic acid in a chewing gum composition and a chewing gum product can be determined by CPC precipitation. In addition, the presence of hyaluronic acid in a chewing gum composition and a chewing gum product can also be determined by conventional and well-known carbazole-sulfuric acid method, HPLC method, ELISA method and the like.

Gum Base

The gum base can be a conventional and well-known gum base, or contain an elastomer (rubber). The elastomer used for a gum base substantially varies in accordance with various factors such as a desired type of a gum base, desired consistency of a gum composition, other ingredients used in a composition for producing an ultimate chewing gum product, and the like. The elastomer can be an arbitrary water-insoluble polymer that is known in the art, including a gum polymer used for chewing gum and bubble gum. A polymer that is suitable for the gum base includes a natural and synthetic elastomer. A non-limiting example of a polymer suitable for use in a gum base composition includes a natural substance (plant-derived) such as chicle, natural gum, crown gum, nispero, rosin, jelutong, perillo, niger gutta, tunu, balata, gutta percha, lechi caspi, sorva, gutta kay and the like, and a combination thereof. A non-limiting example of the synthetic elastomer includes styrene-butadiene copolymer (SBR), polyisobutylene, isobutylene-isoprene copolymer, polyethylene, polyvinyl acetate and the like, and a combination thereof.

In addition, examples of useful polymers include cross-linked polyvinylpyrrolidone, polymethylmethacrylate, copolymer of lactic acid, polyhydroxyalkanoate, plasticized ethyl cellulose, polyvinyl acetate phthalate, and a combination thereof.

The amount of elastomer used in a gum base can be changed in accordance with various factors such as a type of gum base used, desired consistency of a gum composition, other ingredients used in a composition for producing a final chewing gum product, and the like. In general, the content of elastomer is about 10% by mass to 60% by mass with respect to the entirety of the gum base.

The gum base can contain one or more waxes. Wax used herein designates a substance in the form of oil and fat (wax ester) having a high melting point, which is an ester of higher fatty acid with a univalent or divalent higher alcohol, And broadly includes neutral fat, higher fatty acid, hydrocarbon, and the like that show properties similar thereto. The wax softens a mixture of polymer and elastomer and increases the elasticity of the gum base. In a case where a wax is present, the melting point of the wax used is generally no greater than about 60° C., and preferably about 45° C. to 55° C. A low melting wax can be a paraffin wax or a microcrystalline wax.

A high melting wax can be used in the gum base in addition to the low melting wax, in an amount no greater than 5% by mass with respect to the gum base. An example of the high melting wax includes bees wax, vegetable wax, candelilla wax, carnauba wax and the like, and a combination thereof.

It should be noted that it is preferable for the gum base according to the present invention to not substantially contain a wax. As used herein, the term “substantially free of wax” refers to a gum base including wax in an amount no greater than 0.5% by mass with respect to the gum base. In a case where a masticator suffers from gingivitis, a tooth is often insufficiently fixed to the gingiva and loose. If such a masticator masticates chewing gum having a high viscosity, the chewing gum may stick to a tooth and the masticator may experience extreme discomfort. Insufficient mastication due to discomfort may not satisfactorily release hyaluronic acid from the chewing gum, and the spread of hyaluronic acid, disinfection and inactivation of germs may be insufficient due to deficient secretion of saliva. Using a gum base that does not substantially contain a wax can provide a satisfactory effect of suppressing gingivitis, by greatly lowering adhesion to a tooth and avoiding the abovementioned problems.

The gum base preferably contains at least one oil and fat, especially a low melting oil. As used herein, the oil and fat broadly includes glycerin ester of fatty acids, and may include at least one kind of fatty oil or fat. The melting point of the oil is preferably about 15° C. to about 60° C., specifically about 15° C. to about 40° C., and more specifically about 30° C. to about 40° C. The low melting oil provides smoothness to a surface of a chewing gum product and ease of mastication by improving the lubricating property thereof. Non-limiting examples of a suitable low melting oil include a hydrogenated vegetable oil such as hydrogenated palm oil, hydrogenated cotton seed oil, cocoa oil, soy oil, peanut oil, cotton seed oil, sunflower seed oil, olive oil, a combination thereof, and the like.

Such an oil and fat is contained in an amount of preferably at least 20% by mass, more preferably at least 25% by mass, and most preferably at least 32% by mass with respect to a mass of the entire gum base. This can provide a satisfactory effect of suppressing gingivitis by greatly lowering adhesion of a chewing gum to a tooth. It should be noted that, although not limited thereto, an upper limit of content of the oil and fat, considering balanced flavor, is preferably no greater than 50% by mass, more preferably no greater than 40% by mass, and most preferably no greater than 35% by mass.

The gum base may contain an elastomer solvent that assists in softening of an elastomer component. Examples of such an elastomer solvent include elastomer solvents known in the art, such as: terpinene resin such as α-pinene polymer or β-pinene polymer; methyl ester, glycerol ester and pentaerythritol ester of rosin; and modified rosin and gums such as hydrogenated, dimerized and polymerized rosin, and mixtures thereof. Examples of an elastomer solvent preferably used herein include pentaerythritol ester of partially hydrogenated wood and gum rosin, pentaerythritol ester of wood and gum rosin, glycerol ester of wood rosin, glycerol ester of partially dimerized wood and gum rosin, glycerol ester of polymerized wood and gum rosin, glycerol ester of tall oil rosin, glycerol ester of wood and gum rosin, partially hydrogenated wood and gum rosin, partially hydrogenated methyl ester of wood and rosin, and the like, and mixtures thereof. An elastomer solvent can be used in an amount of about 2% by mass to 15% by mass, and more specifically about 7% by mass to 11% by mass with respect to a mass of gum base.

A gum base composition can contain polyvinyl acetate. Polyvinyl acetate, especially polymeric polyvinyl acetate, acts as a filler and can maintain the integrity of the gum base when being masticated. Although polyvinyl acetate of a molecular weight of about 10000 to about 60000 is obtained to be used, combining polyvinyl acetate of different molecular weights and, for example, combining polyvinyl acetate of higher molecular weight and polyvinyl acetate of lower molecular weight, are included in the present embodiment. The molecular weight of a useful polyvinyl acetate of low-molecular weight may be about 10000 to about 15000. The molecular weight of a useful polyvinyl acetate of middle-molecular weight may be about 15000 to about 55000. The molecular weight of a useful polyvinyl acetate of high-molecular weight may be about 50000 to about 100000, and more specifically about 80000 to about 100000. The molecular weight of a useful polyvinyl acetate of ultra-high molecular weight may be at least about 100000. The content of polyvinyl acetate may be about 0% by mass to about 50% by mass, and more specifically about 10% by mass to about 35% by mass, with respect to a mass of the entire gum base.

The gum base can contain an emulsifier that assists dispersion of an immiscible component so as to obtain a uniform, stable state. Examples of a useful emulsifier for the present invention include glyceryl monostearate, lecithin, fatty acid monoglyceride, diglyceride, propylene glycol monostearate and the like, and mixtures thereof. An emulsifier can be used in an amount of about 2% by mass to 15% by mass, and more specifically about 7% by mass to 11% by mass, with respect to a mass of the gum base.

The gum base can contain a plasticizer or softener so as to obtain various desired textures and consistencies. The plasticizer and the softener have a low molecular weight and can penetrate into a basic structure of the gum base, thereby plasticizing and lowering viscosity of the gum base. Examples of a useful plasticizer and a useful softener include lanolin, palmitic acid, oleic acid, stearic acid, sodium stearate, potassium stearate, glyceryl triacetate, glyceryl lecithin, glyceryl monostearate, propylene glycol monostearate, acetylated monoglyceride, glycerine and the like, and mixtures thereof. Waxes, for example, natural and synthetic waxes, hydrogenated vegetable oils, petroleum waxes such as polyurethane waxes, polyethylene waxes, paraffin waxes, microcrystalline waxes, fatty waxes, sorbitan monostearate, tallow, propylene glycol, mixtures thereof, and the like, can also be incorporated into the gum base compositions. The plasticizer and the softener are used in the gum base in an amount of no greater than about 20% by mass, and more specifically about 9% by mass to about 17% by mass, with respect to a mass of the gum base.

The plasticizer includes hydrogenated vegetable oil such as soy oil, cotton seed oil and the like, which can be used singly or in combination. The plasticizers can provide the gum base with a superior texture and a soft chewing property. Generally, the plasticizer and the softener can be used in an amount of about 5% by mass to about 14% by mass, and more preferably about 5% by mass to about 13.5% by mass, with respect to a mass of the gum base.

Commercially available anhydrous glycerin of United States Pharmacopeia (USP) grade and the like can also be used as the softener. Glycerin is a syrup-like liquid with a sweet and agreeable taste, which has a sweetness of about 60% that of cane sugar. Since glycerin is hygroscopic, the anhydrous glycerin can be kept in an anhydrous condition while preparing the chewing gum composition.

In an embodiment, the gum base of the present invention can contain an effective amount of bulking agent such as mineral adjuvant, which can act as a filler and a texture improver. Examples of a useful mineral adjuvant includes calcium carbonate, magnesium carbonate, alumina, aluminum hydroxide, aluminum silicate, talc, tricalcium phosphate, dicalcium phosphate, calcium sulfate and the like, as well as mixtures thereof. Various amounts of the filler or the adjuvant can be used in the gum base. The content of the filler can be about 0% by mass to about 40% by mass, and more specifically about 0% by mass to about 30% by mass, with respect to a mass of the gum base. In an embodiment, the content of the filler can be about 0% by mass to about 15% by mass, and more specifically about 3% by mass to about 11% by mass.

An effective amount of various conventional additives, such as a coloring agent, an antioxidant, preservatives, a flavoring agent and the like, can be optionally blended with the gum base. For example, titanium dioxide and other artificial or natural coloring agents suitable for food, drug and cosmetic applications, known as F. F. & C. dyes, can be used. An antioxidant such as dibutylated hydroxytoluene, butylated hydroxyanisole, propyl gallate, t-butylated hydroquinone, other well-known antioxidant component, mixtures thereof, and the like can also be contained. Other conventional chewing gum additives known to one having ordinary skill in the chewing gum art can also be used in the gum base.

Generally, the content of the gum base can be about 5% by mass to about 95% by mass, and more specifically about 20% by mass to about 60% by mass, with respect to a mass of the entire chewing gum composition.

Optional Components

The chewing gum composition according to the present invention preferably further contains at least one sweetener and casein phosphopeptide-amorphous calcium phosphate complex (CPP-ACP).

Sweetener

A preferred sweetener is a sugarless sweetener and, more specifically, a sweetener that can be selected from a variety of substances such as water-soluble sweeteners, water-soluble artificial sweeteners, water-soluble sweeteners derived from naturally occurring water-soluble sweeteners, dipeptide based sweeteners, and protein based sweeteners, as well as mixtures thereof, and the like. It should be noted that “sugarless” as used herein indicates that the sugar content per 100 g of chewing gum is less than 0.5 mg.

Without being limited to particular sweeteners, representative categories and examples include:

(a) water-soluble sweetening agents such as dihydrochalcones, monellin, steviosides, steviol glycosides, isomers of steviol glycosides, stevia-derived sweeteners, rebaudiosides, rebaudioside A, glycyrrhizin, dihydroflavenol, and sugar alcohols such as sorbitol, mannitol, maltitol, and L-aminodicarboxylic acid aminoalkenoic acid ester amides, such as those disclosed in U.S. Pat. No. 4,619,834, the disclosure of which is incorporated herein by reference, and mixtures thereof;

(b) water-soluble artificial sweeteners such as soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (Acesulfame-K), the free acid form of saccharin, and mixtures thereof;

(c) dipeptide based sweeteners, such as L-aspartic acid derived sweeteners, such as L-aspartyl-L-phenylalanine methyl ester (Aspartame) and materials described in U.S. Pat. No. 3,492,131, L-α-aspartyl-N-(2,2,4,4-tetramethyl-3-thietanyl)-D-alaninamide hydrate (Alitame), N-[N-(3,3-dimethylbutyl)-L-α-aspartyl]-L-phenylalanine 1-methyl ester (Neotame), methyl esters of L-aspartyl-L-phenylglycerine and L-aspartyl-L-2,5-dihydrophenyl-glycine, L-aspartyl-2,5-dihydro-L-phenylalanine, L-aspartyl-L-(1-cyclohexen)-alanine, and mixtures thereof;

(d) water-soluble sweeteners derived from naturally occurring water-soluble sweeteners, such as chlorinated derivatives of ordinary sugar (sucrose), e.g., chlorodeoxysugar derivatives such as derivatives of chlorodeoxysucrose or chlorodeoxygalactosucrose, known, for example, under the product designation of sucralose; examples of chlorodeoxysucrose and chlorodeoxygalactosucrose derivatives include, but are not limited to: 1-chloro-1′-deoxysucrose; 4-chloro-4-deoxy-α-D-galactopyranosyl-α-D-fructofuranoside, or 4-chloro-4-deoxygalactosucrose; 4-chloro-4-deoxy-α-D-galactopyranosyl-1-chloro-1-deoxy-β-D-fructofuranoside, or 4,1′-dichloro-4,1′-dideoxygalactosucrose; 1′,6′-dichloro1′,6′-dideoxysucrose; 4-chloro-4-deoxy-α-D-galactopyranosyl-1,6-dichloro-1,6-dideoxy-β-D-fructofuranoside, or 4,1′,6′-trichloro-4,1′,6′-trideoxygalactosucrose; 4,6-dichloro-4,6-dideoxy-α-D-galactopyranosyl-6-chloro-6-deoxy-β-D -fructofuranoside, or 4,6,6′-trichloro-4,6,6′-trideoxygalactosucrose; 6,1′,6′-trichloro-6,1′,6′-trideoxysucrose; 4,6-dichloro-4,6-dideoxy-α-D-galacto-pyranosyl-1,6-dichloro-1,6-di deoxy-β-D-fructofuranoside, or 4,6,1′,6′-tetrachloro-4,6,1′,6′-tetradeoxygalacto-sucrose; and 4,6,1′,6′-tetradeoxy-sucrose, and mixtures thereof; and

(e) protein based sweeteners such as thaumatococcus danielli (Thaumatin I and II).

An intense sweetener may be used in many distinct physical forms well-known in the art to provide an initial burst of sweetness and/or a prolonged sensation of sweetness. Without being limited thereto, such physical forms include free forms, such as spray dried, powdered, beaded forms, encapsulated forms, and mixtures thereof.

The sweetener can be an intense sweetener, such as sucralose, saccharin salt, acesulfame potassium salt, aspartame, thaumatin, neotame, alitame, as well as combinations thereof and the like.

Casein Phosphopeptide-Calcium Phosphate Complex (CPP-ACP)

CPP-ACP is functional ingredient and an anti-decay agent and the content thereof can be arbitrarily set. According to an embodiment, the content of CPP-ACP may be no greater than 150 mg, no greater than 130 mg, no greater than 110 mg, no greater than 80 mg, no greater than 50 mg, no greater than 20 mg, no greater than 10 mg, no greater than 5 mg, no greater than 1 mg, no greater than 0.5 mg, or no greater than 0.1 mg per serving (per one intake). According to another embodiment, the content of CPP-ACP may be in a range of 0.01 to 150 mg, 0.3 to 80 mg, 0.2 to 20 mg, 2 to 20 mg, or 5 to 20 mg. Here, 1 serving may consist of 1 to 8 pieces of the product, namely 7, 6, 5, 4, 3, or 2 pieces, although not particularly limited thereto. In addition, the mass of each product may be in a range of 1 to 40 g, namely 30 g, 20 g, 10 g, 8 g, 6 g, 4 g, 2 g, 1 g, or 0.5 g, although not particularly limited thereto.

According to an embodiment, the content of CPP-ACP may be no greater than 300 mg, no greater than 200 mg, no greater than 150 mg, no greater than 100 mg, no greater than 50 mg, no greater than 25 mg, no greater than 10 mg, no greater than 5 mg, or no greater than 1 mg per day. According to an embodiment, the content of CPP-ACP can be in a range of 75 to 300 mg, 100 to 200 mg, or 75 to 100 mg per day. As used herein, the amount per day can include a desired arbitral number of servings or products.

According to an embodiment, the content of CPP-ACP may be no greater than 95%, no greater than 90%, no greater than 85%, no greater than 80%, no greater than 75%, no greater than 70%, no greater than 65%, no greater than 60%, no greater than 55%, no greater than 50%, no greater than 45%, no greater than 40%, no greater than 35%, no greater than 30%, no greater than 25%, no greater than 20%, no greater than 15%, no greater than 10%, no greater than 5%, no greater than 2%, no greater than 1%, no greater than 0.5%, no greater than 0.2%, or no greater than 0.1% with respect to the mass of the product. According to another embodiment, the content of CCP-ACP may be in a range of 0.01 to 95%, 0.01 to 60%, 0.01 to 25%, 0.01 to 5%, or 0.01 to 2% with respect to the mass of the product.

At least a part of CPP-ACP is preferably in a form having a modified releasing property, such as being encapsulated. Generally, CPP-ACP is released in a controlled manner by encapsulating at least a part thereof, and thus effects thereof can be sustained over an extended time period.

CPP-ACP can be used either in a state of being encapsulated or in a state of not being encapsulated (free state). In a center-filled gum, encapsulated CPP-ACP can be present in a central filled region and/or a coating region, and non-encapsulated CPP-ACP can be present in the central filled region. The encapsulated CPP-ACP and the non-encapsulated CPP-ACP can be used in an equivalent amount or different amounts.

The capsule material for CPP-ACP includes at least one water-insoluble polymer, copolymer and other materials that can form a solid or rigid coating or film as a protection barrier or a protection layer around at least one component, and/or can form a matrix along with at least one component. In an embodiment, the capsule material can completely enclose, cover, coat, or encapsulate components. In another embodiment, the capsule material can partially enclose, cover, coat, or encapsulate components. Different capsule materials can provide different releasing rates or releasing profiles for the components being encapsulated.

Examples of a useful capsule material include polyvinyl acetate, polyethylene, cross-linked polyvinylpyrrolidone, polymethylmethacrylate, polylactate, polyhydroxyalkanoic acid, ethyl cellulose, polyvinyl acetate phthalate, polyethylene glycol ester, methacrylic acid-co-methyl methacrylate, ethylene vinyl acetate (EVA) copolymer, and combinations thereof.

Flavoring

Examples of suitable flavoring may include those flavors known to skilled artisan, such as natural and artificial flavoring. The flavoring may be chosen from synthetic flavor oils and flavoring aromatics and/or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Other useful flavorings are artificial, natural and synthetic fruit flavors such as vanilla, and citrus oils including lemon, orange, lime, grapefruit and the like, and fruit essences including apple, pear, peach, grape, blueberry, strawberry, raspberry, cherry, plum, pineapple, apricot, banana, melon, apricot and so forth. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath freshening properties, with mint flavors given as a representative example.

Aldehydes and esters such as cinnamyl acetate, cinnamaldehyde, citral diethylacetal, dihydrocarvyl acetate, eugenyl formate, p-methylamisol, and so forth are given as other useful flavoring that may be used. Generally, any flavoring or food additive such as those described in Chemicals Used in Food Processing, publication 1274, pages 63-258, by the National Academy of Sciences, may be used. This publication is incorporated herein by reference. Either natural or artificial flavoring can be used.

Further examples of aldehyde flavorings include, but are not limited to, acetaldehyde (apple), benzaldehyde (cherry, almond), anisic aldehyde (licorice, anise), cinnamic aldehyde (cinnamon), citral, i.e. α-citral (lemon, lime), neral, i.e. β-citral (lemon, lime), decanal (orange, lemon), ethyl vanillin (vanilla, cream), heliotrope, i.e. piperonal (vanilla, cream), vanillin (vanilla, cream), α-amyl cinnamaldehyde (balmy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e. trans-2 hexarial (berry fruits), tolyl aldehyde (cherry, almond), veratraldehyde (vanilla), 2,6-dimethyl-5-heptenal, i.e. melonal (melon), 2,6-dimethyloctanal (unripe fruit), and 2-dodecenal (citrus, mandarin), cherry, grape, strawberry, shortcake, and mixtures thereof.

In an embodiment, the flavoring may be employed in either liquid form and/or dried form. In a case where employing in the latter form, a suitable drying means such as spray drying of the oil may be used. Alternatively, the flavoring may be absorbed onto water soluble materials, such as cellulose, starch, sugar, maltodextrin, gum arabic and so forth or may be encapsulated. The actual techniques for preparing such dried forms are well-known.

In an embodiment, the flavoring may be used in many distinct physical forms. Without being limited thereto, such physical forms include free forms, such as spray dried, powdered, beaded forms, encapsulated forms, and mixtures thereof.

The amount of flavor employed herein may be a matter of preference subject to such factors as the individual flavoring and the strength of the flavor desired. Therefore, the amount of flavoring may be varied in order to obtain the result desired in the final product. Such a variation is within the skill of those in the art, and does not require much experiment. In general, the flavoring is present in amounts of about 0.02% to about 5%, more specifically from about 0.1% to about 2%, and even more specifically, from about 0.8% to about 1.8%, by mass of the chewing gum composition.

Drugs

The chewing gum composition of the present invention can contain various drugs such as medicaments, herbals, nutritional supplements and the like. Examples of useful drugs include ACE-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies such as sildenafil citrate, which is currently marketed as Viagra (registered trademark), fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids such as bromocryptine or nicotine, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, cardiotonic agents, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, uterine relaxants, anti-obesity drugs, erythropoietic drugs, anti-asthmatics, cough suppressants, mucolytics, DNA and genetic modifying drugs, and combinations thereof. More specifically, the chewing gum composition can contain, although not particularly limited thereto, delmopinol in an amount of about 0.5 to 1.0% by mass with respect to a mass of the entire composition. In addition, in order to further suppress gingivitis, the chewing gum composition can contain LAE (ethyl ester of the lauramide of arginine hydrochloride) represented by the following formula in an amount of about 0.006 to 0.015% by mass.

Indicia

The chewing gum product can include an indicia indicating the effectiveness for therapy, prophylaxis, or retardation of progression of gingivitis. The indicia can include a visual, aromatic, or organoleptic cue. As used herein, “therapy” refers to an improvement or suppression of worsening, at least in part, of symptoms of gingivitis, by using the chewing gum product after an onset thereof. “Prophylaxis” refers to suppression of the onset of a part of, or all of, the symptoms of gingivitis, by using the chewing gum product before the onset. Furthermore, “retardation of progression” indicates that the speed at which symptom development of gingivitis is decreased compared to a case where the chewing gum product of the present invention is not used.

Preparation Method

The chewing gum composition may be prepared using standard techniques and equipment known to those skilled in the art. A useful apparatus in accordance with the embodiments described herein includes a mixing apparatus and a heating apparatus well known in the chewing gum manufacturing arts. Therefore, selection of a specific apparatus is apparent to those skilled in the art. For general chewing gum preparation processes, see U.S. Pat. Nos. 4,271,197 to Hopkins et al, 4,352,822 to Cherukuri et al and 4,497,832 to Cherukuri et al, each of which is incorporated herein by reference in its entirety.

More specifically, a dispersed material of components can be formed by blending a gum base with hyaluronic acid or a physiologically acceptable salt thereof. More particularly, the gum base can be melted in a high-shear mixer at a high temperature. Optional components and hyaluronic acid or a physiologically acceptable salt thereof are added to the melted gum base and blended in high-shear, thereby entirely dispersing the compositions. The components can be blended at a high temperature of about 50 to 150° C. A mixture of the components that is obtained can be cooled.

Here, hyaluronic acid or a physiologically acceptable salt thereof is preferably added in a last half of a process of blending other components. This can facilitate appropriate release of hyaluronic acid or a physiologically acceptable salt thereof during mastication. As used herein, “a last half of a process of blending other components” can refer to a period after which half of the total amount of other components has been added, or a period after which half of a time period for adding other components has elapsed. In addition, the amount of hyaluronic acid or a physiologically acceptable salt thereof to be added can be continuously or intermittently increased over time. It should be noted that, as used herein, “continuously or intermittently increased” indicates that the amount tends to be increased as a whole, and includes an aspect where a phase of decreasing the amount to be added is momentarily inserted for circumvention.

For example, center-filled chewing gum embodiments may include a central filled region, which may be a liquid or powder or other solid, and a coating region. The coating region may at least partially surround the gum region. Hyaluronic acid or a physiologically acceptable salt thereof can be disposed in any region of the center-filled chewing gum: in other words, in the central filled region, the gum region, and/or the coating region. Center-filled chewing gums and methods of preparing the same are more completely described in assignee's co-pending U.S. patent application Ser. No. 10/925,822, filed on Aug. 24, 2004 and assignee's co-pending U.S. patent application Ser. No. 11/210,954, filed on Aug. 24, 2005, both entitled “Liquid-Filled Chewing Gum Composition,” the contents both of which are incorporated herein by reference.

Some other embodiments of a chewing gum include, for example, a compressed gum style such as a pressurized tablet gum and the like. Such embodiments can include a fine-grained chewing gum base. An example of a fine-grained chewing gum base includes a compressible gum base composition and a powder for tableting. The compressed chewing gum is more completely described in assignee's co-pending U.S. Patent Application No. 60/734,680, filed on Nov. 8, 2005, entitled “Compressible Gum System,” the contents of which are incorporated herein by reference.

In some embodiments, the chewing gum may have a coating thereon. Such coated chewing gums are typically referred to as pellet gums. The outer coating may be hard or crunchy. Any suitable coating material known to those skilled in the art may be employed. Typically, the outer coating may include sorbitol, maltitol, xylitol, isomalt, erythritol and other crystallizable polyols; sucrose may also be used. Furthermore, the coating may include several opaque layers, such that the chewing gum composition is not visible through the coating itself, which can optionally be further covered with one or more transparent layers for aesthetic, textural and protective purposes. The outer coating may also contain small amounts of water and gum arabic. The coating can be further coated with wax. The coating may be applied in a conventional manner by successive applications of a coating solution, with drying in between each coat. As the coating dries, it usually becomes opaque and is usually white, although other colorants may be added. A polyol coating can be further coated with wax. The coating can further include colored flakes or speckles. If the composition includes a coating, it is possible that one or more oral care active ingredients can be dispersed throughout the coating. This is especially preferred if one or more oral care active ingredients is incompatible in a single phase composition with another of the active ingredients. Flavoring may also be added to yield unique product characteristics.

Other materials may be added to the coating to achieve the desired properties. These materials may include without limitations, cellulosics such as carboxymethyl cellulose, gelatin, xanthan gum and gum arabic.

The coating composition may be applied by any method known in the art including the method described above. The coating composition may be present in an amount from about 2% to about 60%, and more specifically from about 25% to about 45%, by mass of the chewing gum product.

Kit

The present invention includes a kit for therapy or prophylaxis for gingivitis in a mammal, including:

a chewing gum product containing a gum base, and hyaluronic acid or a physiologically acceptable salt thereof;

an instructional insert for the chewing gum product; and

a package for encasing the chewing gum product and the instructional insert.

In addition, the present invention further includes a kit for retardation of progression of gingivitis in a mammal, including:

a chewing gum product containing a gum base, and hyaluronic acid or a physiologically acceptable salt thereof;

an instructional insert for the chewing gum product; and

a package for encasing the chewing gum product and the instructional insert.

Mode of Usage

The chewing gum product according to the present invention may be masticated with arbitral frequency for an arbitral period of time in an arbitral amount, in consideration of hyaluronic acid content, degree of gingivitis and the like. More specifically, in terms of sufficiently realizing therapy, prophylaxis, or retardation of progress of gingivitis, it is preferable that the chewing gum product is masticated in an amount equivalent to at least 4 mg of hyaluronic acid per day. On the other hand, in consideration of psychological stress of a masticator, it is preferable that, generally, 1 to 8 pieces (one serving) of the chewing gum product is masticated one to four times a day.

### EXAMPLES

### Example 1

A gum base was prepared according to the composition shown in Table 1. More specifically, the gum base was prepared by adding, heating and melting the components shown in Table 1 in a mixer.

TABLE 1

| Components | % by mass |
|---|---|
| Elastomer | 8-15 |
| Resin ester | 5-10 |
| Polyvinyl acetate | 20-30 |
| Wax | 0.0-0.5 |
| Fat & Oil | 20-40 |
| Emulsifying agent | 5-10 |
| Filler | 10-25 |

A plate-like chewing gum product was manufactured by blending: hyaluronic acid with an average molecular weight of 800000 “HA-F” (manufactured by Q. P. Corporation); an appropriate amount of a well-known sugarless sweetener; and an appropriate amount of flavoring, so that content of hyaluronic acid in the chewing gum product is 10 mg per 1 g.

### Example 2

A chewing gum product was manufactured by a similar procedure to that for Example 1, except for using hyaluronic acid having an average molecular weight of 400 manufactured according to a conventional method.

### Example 3

A chewing gum product was manufactured by a similar procedure to that for Example 1, except for using hyaluronic acid having an average molecular weight of 10000000 manufactured according to a conventional method.

Releasing Property Assessment

10 mM phosphate buffer (pH 7.3) containing 145 mM sodium chloride was added to a chewing gum product of the same composition to that of Example 1, except for not containing hyaluronic acid, thereby extracting a control gum solution. To the control gum solution thus obtained, the hyaluronic acid used for Example 1 was added so that the final contents thereof were 450, 225, 112.5 and 56.25 μg/ml, thereby preparing hyaluronic acid-doped control gum solutions. The solutions were filtered through a membrane filter having a pore diameter of 0.45 μm, and the solutions thus filtered were retrieved as hyaluronic acid reference solutions. For each hyaluronic acid reference solution, absorbance was determined at a wavelength of 208 nm, and a correlation equation between hyaluronic acid content and absorbance. A correlation factor R2 of the correlation equation, 0.9947, was extremely high.

y=0.0013x+0.0826 Correlation Equation

(wherein x is hyaluronic acid content (4 g/ml), and y is absorbance)

A hyaluronic acid extract was retrieved by using the chewing gum product prepared in Example 1. For the extract, absorbance was determined in the abovementioned procedure, and the amount of hyaluronic acid released from the chewing gum product was calculated based on a value thus determined and the correlation equation. As a result, the amount of released hyaluronic acid per 1 g was 9.53 mg. The ratio of the released amount with respect to the content (releasing ratio) was determined to be 95.3% (mass/mass), which was extremely high.

The amount of hyaluronic acid released was calculated in relation to the chewing gum product of Examples 2 and 3, in a similar procedure. As a result, the amount of hyaluronic acid released in relation to Example 2 was 9.73 mg, and the ratio of the amount released with respect to the content (releasing ratio) was confirmed to be 97.3% (mass/mass), which was high. In addition, the amount of hyaluronic acid released for the chewing gum product of Example 3 was 9.35 mg, and the ratio of the amount released with respect to the content (releasing ratio) was determined to be 93.5% (mass/mass), which was moderately high.

### Examples 4 and 5

A tablet shaped chewing gum product was manufactured with the hyaluronic acid content being 1 mg (Example 4) and 5 mg (Example 5) per piece of chewing gum product (1.5 g), and using 2.3 mg of CPP-ACP, an appropriate amount of gum arabic, and wax. Materials used were same as in Example 1.

### Examples 6 and 7

Chewing gum products were manufactured according to the same procedure as that of Examples 4 and 5, except for a gum base being prepared according to a composition shown in Table 2.

TABLE 2

% by mass

| Components | Example 6 | Example 7 |
|---|---|---|
| Elastomer | 5-10 | 10-15 |
| Resin ester | 15-20 | 15-20 |
| Polyvinyl acetate | 20-25 | 20-25 |
| Wax | 5-10 | 5-10 |
| Fat & Oil | 5-10 | 20-25 |
| Emulsifying agent | 5-10 | 5-10 |
| Filler | 20-25 | 10-15 |

Clinical Evaluation

Regarding Examples 4 and 5, the gingivitis improvement effect of the chewing gum products was determined by dental examination. 74 healthy subjects (men and women) of age 18 to 65, diagnosed as having plaque-induced gingivitis or light periodontitis in accordance with Shishubyo No Shindan To Chiryo No Gaidorain (Guideline for Diagnostics and Treatment of Periodontal Disease) edited by Shakai Hoken Kenkyujo, were randomly divided into three groups: a group taking Example 4 (n=23); a group taking Example 5 (n=25); and a group taking Comparative Example 1 (similar to Example 4 except for not containing hyaluronic acid) (n=26). A double-blind intake trial was conducted for a two week period. Intake of the chewing gum products was performed by masticating two pieces of the tablet gum at once, for about 20 minutes, four times a day. Intake was performed after breakfast, after lunch, after dinner, and one to two hours before bedtime. The post-meal intake was performed after eating or, in a case where a subject had a habit of oral cleaning after meals, after the oral cleaning. Mastication before bedtime was performed at least one hour before oral cleaning at bedtime. The subjects were not advised of a particular masticating method, and were forbidden to eat and drink at least one hour after mastication.

The dental examination of gingivitis was conducted by a periodontist, with an index of bleeding from gingiva in a probing using a pressure-sensitive pocket probe (Bleeding Index: BI) and a ratio of the number of bleeding regions with respect to the number of probed regions (Percent of Bleeding on Probing: % BOP) as a clinical indicator indicating a condition of gingivitis

All teeth were subjected to the probing and 6 regions were probed for each tooth (mesiobuccal region, buccal center, distobuccal region, mesiolingual region, lingual center, and distolingual region). A BI score regarding the presence of bleeding while probing was determined for each region in accordance with the following criteria.

Score 0: No bleeding

Score 1: Bled 30 seconds after probing

Score 2: Bled immediately after probing

Regions with Score 1 and Score 2 were considered to be positive regions of bleeding, and the percent of the positive regions with respect to probed regions was expressed as % BOP. The differences in scores obtained at the beginning of the trial and two weeks after starting intake were obtained, and then an average value of BI and % BOP was obtained for each region. The results thereof are shown in Table 3. The significance of the difference between the groups was determined by using a Mann-Whitney test, with a condition of p<0.05.

TABLE 3

| | Difference in scores obtained in probed regions at start-up | | | | | |
|---|---|---|---|---|---|---|
| | of trial and 2 weeks after starting intake | | | | | |
| | Back Tooth (Molar) | | | | | |
| | BI | | | % BOP | | |
| | Example | Example | Comparative | Example | Example | Comparative |
| Dental | 4 | 5 | Example 1 | 2 | 3 | Example 1 |
| Region | (n = 23) | (n = 25) | (n = 26) | (n = 23) | (n = 25) | (n = 26) |
| Left | 0.35 | 0.35* | 0.04 | 0.20* | 0.20* | 0.03 |
| maxillary | | | | | | |
| buccal | | | | | | |
| Left | 0.37* | 0.37* | −0.04 | 0.22* | 0.19* | −0.02 |
| mandibular | | | | | | |
| buccal | | | | | | |
| Left teeth | 0.39* | 0.27 | 0.13 | 0.21* | 0.15 | 0.07 |
| Left teeth | 0.36* | 0.36* | 0.01 | 0.21* | 0.20* | 0.01 |
| buccal | | | | | | |
| Mandibular | 0.40* | 0.32 | 0.18 | 0.21* | 0.17 | 0.10 |
| teeth | | | | | | |
| Maxillary | 0.31 | 0.39* | 0.10 | 0.18 | 0.21* | 0.05 |
| buccal | | | | | | |
| Teeth | | | | | | |
| Mandibular | 0.37* | 0.38 | 0.09 | 0.20* | 0.19 | 0.04 |
| buccal | | | | | | |
| teeth | | | | | | |

*Significantly different from Comparative Example 1 (p < 0.05)

As shown in Table 3, for the chewing gum product of Examples 4 and 5, almost all dental regions had significantly high BI and % BOP compared to Comparative Example 1. This shows that chewing gum products containing hyaluronic acid can sufficiently suppress gingivitis.

Sensory Assessment

During the abovementioned clinical trial, psychological stress associated with mastication of chewing gums of Examples 4 to 7, of the abovementioned Comparative Example 1, and of Comparative Example 2 (similar to Example 6 except for not containing hyaluronic acid) was assessed. The assessment was conducted after each mastication, in accordance with the following criteria. Average values of scores obtained from the subjects in two weeks are shown in Table 4.

Score 0: Mastication could not be sufficiently performed due to a strong sensation of loose teeth caused by adhesion of the chewing gum product to teeth

Score 1: Subject felt stressed due to a slight sensation of loose teeth caused by adhesion of the chewing gum product to teeth

Score 2: Subject did not feel any stress

TABLE 4

| | | Example | Example | Example | Example | Comparative | Comparative |
|---|---|---|---|---|---|---|---|
| | | 4 | 5 | 6 | 7 | Example 1 | Example 2 |
| Score | 0th to | 1.1 | 1.2 | 0.7 | 0.7 | 0.9 | 0.7 |
| | 2nd day | | | | | | |
| | 3rd to | 1.4 | 1.4 | 0.9 | 1.0 | 0.8 | 0.4 |
| | 7th day | | | | | | |
| | 8th to | 1.8 | 1.8 | 1.0 | 1.3 | 0.7 | 0.3 |
| | 14th day | | | | | | |

As shown in Table 4, the chewing gum product of Comparative Examples 1 and 2 had a lower score and psychological stress increased over time. This is assumed to have been caused by amplification of the stress and progress of gingivitis due to repeated mastication.

On the contrary, with the chewing gum products of Examples 4 to 7, psychological stress was lowered over time. Especially from the second week of intake, subjects felt very little stress. This shows that chewing gum products containing hyaluronic acid can provide hyaluronic acid to an affected area continuously, with little psychological stress.

In addition, Examples 4 and 5 having higher scores than Examples 6 and 7 show that, by using a gum base that does not substantially contain wax, a chewing gum can provide hyaluronic acid to an affected area continuously with less psychological stress. It should be noted that, although Comparative Example 1, which uses the same gum base as in Example 4, had scores higher than Examples 6 and 7 for 2 days after starting mastication, gingivitis progressed due to the absence of hyaluronic acid, and thus the scores became lower than Examples 6 and 7 from the third day after starting mastication.

Furthermore, Example 7, which has a higher score than Example 6, shows that chewing gum containing fat and oil in an amount of at least 20% with respect to a mass of the entire gum base can provide hyaluronic acid to an affected area continuously with even less psychological stress.

### Example 8

A chewing gum product that is securely hardened and contains 1.2 g of hyaluronic acid per piece (1.5 g) was prepared by: packing powder, which was obtained by blending 1.2 g of hyaluronic acid, an appropriate amount of gum base, a well-known sugarless sweetener, flavoring, a binding agent and a lubricant, in a tube body; and tableting (Example 8). Other procedures are similar to that of Example 4.

### Example 9

A chewing gum product containing 0.6 g of hyaluronic acid was prepared by similar procedures to that for Example 8, except for the filling amount of hyaluronic acid being changed from 1.2 g to 0.6 g.

### Example 10

A chewing gum product containing 0.15 g of hyaluronic acid was prepared by a similar procedures to that for Example 8, except for the filling amount of hyaluronic acid being changed from 1.2 g to 0.15 g.

A sensory assessment was carried out regarding the texture during mastication of the chewing gum products prepared in Examples 8 to 10, by 10 healthy subjects (men and women) of age 18 to 65, diagnosed as having plaque-induced gingivitis or light periodontitis. The assessment was made in accordance with the following criteria. The results thereof are shown in Table 5.

Score 1: Bad texture due to melted gum base

Score 2: Normal texture as conventional chewing gum products

Score 3: Superior texture over conventional chewing gum products

TABLE 5

| | Example 8 | Example 9 | Example 10 |
|---|---|---|---|
| Average Score | 1.1 | 1.4 | 2.8 |

As shown in Table 5, Example 10 can provide an extremely superior texture over Examples 8 and 9. This shows that texture can be remarkably improved by making the content of hyaluronic acid no greater than 10% by mass with respect to the mass of the composition.

1. A chewing gum composition, comprising a gum base; and a gingivitis-inhibiting amount of hyaluronic acid or a physiologically acceptable salt thereof.

2. The chewing gum composition of claim 1, wherein the hyaluronic acid has an average molecular weight of at least 400 and no greater than 10000000.

3. The chewing gum composition of claim 1, wherein the gingivitis-inhibiting amount is at least 0.0075% by mass and no greater than 95% by mass with respect to a mass of the entire composition.

4. The chewing gum composition of claim 1, wherein the gum base contains fat and oil in an amount of at least 20% by mass with respect to a mass of the entire gum base.

5. The chewing gum composition of claim 1, wherein the gum base is substantially free from a wax.

6. The chewing gum composition of claim 1, further comprising at least one sweetener; and casein phosphopeptide-calcium phosphate complex in an amount of at least 0.01% by mass and no greater than 95% by mass with respect to the mass of the composition.

7. The chewing gum composition of claim 6, wherein at least a part of the casein phosphopeptide-calcium phosphate complex is encapsulated.

8-9. (canceled)

10. (canceled)

11. The chewing gum composition of claim 1, which comprises at least 0.2 mg and no greater than 560 mg of hyaluronic acid or a physiologically acceptable salt thereof.

12. The chewing gum composition of claim 1, which is plate-like, slab, powdered, liquid, paste, pellet, rod-like, center-filled, deposited, or pressed.

13. The chewing gum composition of claim 1, further comprising a central filled region; and a coating region that at least partially coats the central filled region, and wherein the hyaluronic acid or the physiologically acceptable salt thereof is disposed in the central filled region, the coating region, or in both the central filed region and the coating region.

14. The chewing gum composition of claim 13, wherein the hyaluronic acid or the physiologically acceptable salt thereof is disposed in the central filled region, the coating region, or in both the central filed region and the coating region.

15-16. (canceled)

17. A method for preparing a chewing gum composition suitable for inhibiting gingivitis, the method comprising blending a gum base with a gingivitis-inhibiting amount of hyaluronic acid or a physiologically acceptable salt thereof.

18. The method of claim 17, wherein the hyaluronic acid or the physiologically acceptable salt thereof is added in a last half of a process of blending other components.

19. The method of claim 17, wherein the hyaluronic acid has an average molecular weight of at least 400 and no greater than 10000000.

20. The method of claim 17, wherein the gingivitis-inhibiting amount is at least 0.0075% by mass and no greater than 95% by mass with respect to a mass of the entire composition.

21. The method of claim 17, further comprising blending fat and oil in a ratio of at least 20% by mass with respect to a mass of the entire gum base.

22. The method of claim 17, wherein a wax is substantially not blended into the gum base.

23. The method of claim 17, further comprising blending, with the gum base, at least one sweetener; and casein phosphopeptide-calcium phosphate complex in a ratio of at least 0.01% by mass and no greater than 95% by mass with respect to the mass of the composition.

24. The method of claim 23, wherein at least a portion of the casein phosphopeptide-calcium phosphate complex is encapsulated.

25-26. (canceled)

27. Use of a chewing gum product comprising the chewing gum composition of claim 1 for the treatment of gingivitis.

28. The use of claim 27, wherein a content of hyaluronic acid or a physiologically acceptable salt thereof is at least 0.2 mg and no greater than 560 mg.

29. The use of claim 27, wherein the chewing gum product is a plate-like shape, a slab shape, a powdered form, a liquid form, a paste form, a pellet form, a rod-like shape, a center-filled form, a deposited form, or a pressed form.

30. The use of claim 27, wherein the chewing gum product comprises a central filled region; and a coating region that at least partially coats the central filled region, and said hyaluronic acid or the physiologically acceptable salt thereof is disposed in the central filled region, the coating region, or in both thereof

31-35. (canceled)
